(19) United States
(12) Patent Application Publication
(10) Pub. No.: US 2022/0072030 A1
(43) Pub. Date: Mar. 10, 2022

(54) PHARMACEUTICAL FORMULATIONS AND COMPOSITIONS SUITABLE TO TREAT MUCOSITIS

(71) Applicants: ASCULAI Samuel, Toronto, Ontario (CA); HUGHES Ivor, Toronto, Ontario (CA)

(72) Inventors: ASCULAI Samuel, Toronto, Ontario (CA); HUGHES Ivor, Toronto, Ontario (CA)

(21) Appl. No.: 17/299,109

(22) PCT Filed: Nov. 26, 2019
(86) PCT No.: PCT/US2019/063143

§ 371 Date: Jun. 2, 2021

Related U.S. Application Data
(60) Provisional application No. 62/774,527, filed on Dec. 3, 2018.

(51) Int. Cl. A61K 31/728; A61K 31/195

(52) U.S. Cl. CPC A61K 31/728; A61K 31/195

(57) ABSTRACT

This invention relates to the treatment of mucositis, including oral mucositis, pharmaceutical formulations and compositions suitable for such treatment, the use of such compositions to treat mucositis and methods of such treatment.

### CROSS-REFERENCE TO RELATED APPLICATIONS

This PCT application claims the benefit under 35 U.S.C. § 119(e) of Application Ser. No. 62/774,527 filed on Dec. 3, 2018 entitled “PHARMACEUTICAL FORMULATIONS AND COMPOSITIONS SUITABLE TO TREAT MUCOSITIS” whose entire disclosure is incorporated by reference.

### FIELD OF INVENTION

This invention relates to the treatment of mucositis, including oral mucositis, pharmaceutical formulations and compositions suitable for such treatment, the use of such compositions to treat mucositis and methods of such treatment.

### BACKGROUND OF THE INVENTION

Mucositis consists of the inflammation of the soft tissue layer (mucous membranes) lining the digestive system from the mouth to the anus. It is a common, serious, and debilitating side effect in the digestive system of radiation and chemotherapy given to, for example, cancer patients. Mucositis can make swallowing, eating, and speaking very difficult for such patients.

Mucositis affects the rapidly dividing mucosal cells that line the mouth, throat, stomach, and intestines. These mucosal cells have a short life and when destroyed may not be replaced immediately by the body. In that event, the resultant inflammation caused in those areas exhibits raw sores and even ulcers in the patient's mouth, throat and digestive system inevitably causing the patient to suffer debilitating pain and puts the patient to greater risk for infection. The resultant inflammation also causes the over expression of the hyaluronan receptors CD44 and RHAMM.

When mucositis occurs in the mouth and esophagus, it is known as oral mucositis. When it occurs in the stomach, it is known as gastrointestinal mucositis.

U.S. Pat. No. 5,972,906 issued Oct. 26, 1999 (Asculai et al) purports to teach the use of exogenous hyaluronic acid formulations for the treatment of mucous membrane trauma disease or condition for the relief of the pain associated therewith and enable the healing thereof. The form of the exogenous hyaluronic acid in the formulations exemplified, has a molecular weight between about 150000 Daltons and about 750000 Daltons. The formulations contain diclofenac.

The formulations exemplified are applied to aphthous ulcers in the patient's mouth and provide relief.

The said patent mentions the use of the formulations for the treatment of “ . . . leukoplakia, (oral) mucositis, burning mouth syndrome, lichen planus, denture sores, gingivitis, recent oral surgical sites, cervical dysplasia, vulva leukoplakia and other vulval lesions, Bechets Syndrome, radiotherapy induced mucositis, post-operative gum pain, traumatic mouth legions, post-radiotherapy vaginitis, non-specific vaginal inflammatory conditions, and other viral auto-immune and inflammatory ulcerations of the oral and vaginal mucosa . . . ”

The patent however only exemplifies the use of the exemplified formulations with the treatment of aphthous ulcers. The area of the aphthous ulcer is taught to be first dried and then a dosage amount of the formulation is applied directly to the aphthous ulcer. The formulation provides rapid pain relief to the patient with the aphthous ulcer(s) while the ulcer heals. The formulation which stays on the ulcer, penetrates the ulcer and provides continuing relief even when the patient eats or drinks.

Mucositis, however, unlike aphthous ulcers is larger in area and extends deeper into the affected tissue. Mucositis requires deep penetration of a suitable formulation to enable pain relief and enable healing and reduce the greater risk of infection. Today patients are even treated using opioid analgesics to ameliorate pain. This attempt has not been successful and is even frowned on.

Large areas of mucositis, to a greater extent, and aphthous ulcers, to a lesser extent, because of the differences in size of each, express CD-44 and RHAMM hyaluronan receptors which will enable treatment with formulations containing exogenous forms of hyaluronic acid. Thus, both mucositis, to some extent, and aphthous ulcers will be treatable with the formulations of U.S. Pat. No. 5,972,906.

However, during the almost 20-year period since the issue of U.S. Pat. No. 5,972,906, there is no mention of the use of, or the marketing of, the formulations of U.S. Pat. No. 5,972,906 for the treatment of mucositis. (U.S. Pat. No. 6,159,955 claims in claim 31, the treatment of, among a list of conditions, mucositis, using the same formulations as U.S. Pat. No. 5,972,906 teaches.)

Thus, despite the teachings of the formulations and the methods of treatment in the two US patents, and the lapse of more than 20 years or so, a successful treatment for mucositis has not been advanced, let alone, become standard in healthcare. The need for such treatment is now even greater than ever.

Of course, the treatments in both patents use the same formulations. For example, formulations comprise 2½% by weight of the form of exogenous hyaluronic acid used which has a molecular weight less than about 750000 Daltons and greater than about 150000 Daltons and about 3% by weight of an NSAID such as diclofenac.

Various molecular weights of the form of exogenous hyaluronic acid are exemplified, in the two patents, such as 679000 Daltons, about 225000, and a range between 150000 to 225000 Daltons.

Reference is also made in the two patents to Canadian Letters Patent 1205031 which refers to hyaluronic acid fractions between about 50000 to 100000 Daltons, 250000 to 350000 Daltons and 500000 to 750000 Daltons.

U.S. Pat. No. 5,442,053 (Della Valle et al) references fractions of hyaluronic acid including the reference to a fraction named by the inventor, HYALASTINE which is said to be non-inflammatory and useful for wound healing and which has an average molecular weight between 50000 and 100000 and which is substantially free of a form of hyaluronic acid having an average molecular weight less than about 30000 Daltons. According to Della Valle et al, a form of hyaluronic acid having a molecular weight less than 30000 Daltons has inflammatory activity.

Suggested uses of the HYALASTINE include use in dermatology to treat diseases of mucous membranes of the oral and nasal cavities and as a vehicle for drugs (basic).

It is therefore an object of this invention to provide for the treatment of mucositis, including oral mucositis, pharmaceutical formulations and compositions suitable for such treatment, the use of the suitable formulations and compositions for the treatment of mucositis, including oral mucositis, and methods of such treatment.

Further and other objects of the invention will be realized by those skilled in the art from the following summary of the invention and detailed discussion thereof.

### SUMMARY OF INVENTION

According to one aspect of the invention, we provide a pharmaceutical composition suitable for use to treat mucositis (including oral mucositis) in an animal (a human, for example) suffering from mucositis, said composition comprising an anti-inflammatory (non-inflammatory) first fraction of a form of exogenous hyaluronic acid selected from hyaluronic acid, a pharmaceutically acceptable salt thereof, a pharmaceutically acceptable ester thereof, a fraction thereof and a subunit thereof, for example, having a molecular weight between about 1200, (for example 2000) Daltons and about 5000 Daltons or, a molecular weight of about 7.5 kDaltons, or between about 16000 Daltons and about 20000 Daltons, and combinations which are anti-inflammatory (non-inflammatory),

and

an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons

the first fraction form of hyaluronic acid having a lower molecular weight than the second fraction form of hyaluronic acid

and the first fraction having a lower viscosity than the second fraction.

According to another aspect of the invention, we provide the use of a pharmaceutical composition suitable to treat mucositis (including oral mucositis) in an animal (for example a human) suffering from mucositis, to treat mucositis by applying an effective amount of the composition to the mucositis until the site of the mucositis is healed, said composition comprising

an anti-inflammatory (non-inflammatory) first fraction of a form of exogenous hyaluronic acid selected from hyaluronic acid, a pharmaceutically acceptable salt thereof, a pharmaceutically acceptable ester thereof, a fraction thereof and a subunit thereof, for example, having a molecular weight between about 1200 (for example 2000) Daltons and about 5000 Daltons or a molecular weight of about 7500 Daltons (7.5 kDaltons) or between about 16000 Daltons and about 20000 Daltons, and combinations which are anti-inflammatory (non-inflammatory),

and

an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons

the first fraction form of hyaluronic acid having a lower molecular weight than the second fraction form of hyaluronic acid

and the first fraction having a lower viscosity than the second fraction.

According to another aspect of the invention, we provide an effective amount of a pharmaceutical composition suitable for use to treat mucositis (for example oral mucositis) in an animal (human, for example) suffering from mucositis, said composition comprising

an anti-inflammatory (non-inflammatory) first fraction of a form of exogenous hyaluronic acid selected from hyaluronic acid, a pharmaceutically acceptable salt thereof, a pharmaceutically acceptable ester thereof, a fraction thereof and a subunit thereof and combinations thereof which are non-inflammatory, (for example having a molecular weight between about 1200 (for example 2000) Daltons and about 5000 Daltons or a molecular weight about 7.5 kDaltons or, between about 16000 Daltons and about 20000 Daltons),

and

an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons

the first fraction form of hyaluronic acid having a lower molecular weight than the second fraction form of hyaluronic acid

and the first fraction having a lower viscosity than the second fraction.

According to another aspect of the invention, we provide a method of treating mucositis, the method comprising applying effective amounts of a pharmaceutical composition suitable for use to treat mucositis (for example, oral mucositis) in an animal (a human, for example) suffering from mucositis until the mucositis is healed,

said composition comprising

an anti-inflammatory (non-inflammatory) first fraction of a form of exogenous hyaluronic acid selected from hyaluronic acid, a pharmaceutically acceptable salt thereof, a pharmaceutically acceptable ester thereof, a fraction thereof and a subunit thereof, for example, having a molecular weight between about 1200 (for example 2000) Daltons and about 5000 Daltons or a molecular weight about 7.5 kDaltons or between about 16000 Daltons and about 20000 Daltons, and combinations which are anti-inflammatory (noninflammatory),

and

an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons

the first fraction form of hyaluronic acid having a lower molecular weight than the second fraction form of hyaluronic acid and the first fraction having a lower viscosity than the second fraction.

The first fraction may constitute between about 0.5 percent and about 3 percent of the pharmaceutical composition. The second fraction may constitute between about 2 percent and about 4 percent of the pharmaceutical composition.

According to another aspect of the invention, the pharmaceutical composition further comprises a pharmaceutically tolerable non-toxic amount of COX-1 and/or COX-2 inhibitor which is not a form of hyaluronic acid. The inhibitor may be selected from diclofenac, diclofenac sodium and another pharmaceutically tolerable salt of diclofenac and other such inhibitors in an effective non-toxic amount.

Amounts of the inhibitor, such as diclofenac sodium, may comprise between about 2½ percent and about 4 percent of the pharmaceutical composition.

As diclofenac, (or a form thereof) when selected as the COX-1 and/or COX-2 inhibitor, has a bitter taste, when the composition is formulated for oral use with diclofenac, the composition preferably contains a Taste Masker to make the composition more palatable.

Taste Maskers are known to persons skilled in the art for this purpose and may be selected from any Taste Maskers suitable for masking the taste of the composition for oral use. Taste Maskers may be selected from non-toxic amounts of non-toxic chemicals, or non-toxic plant or fruit extracts as is known in the art.

### DISCUSSION OF THE USE OF THE INVENTION

The first fraction of the form of hyaluronic acid, being less viscous than the second fraction, will act not only as a vehicle for the first fraction and the inhibitor, such as diclofenac, but also, will bind with the expressed receptors for hyaluronic acid of the mucositis which because of the inflamed tissue in the mucositis, the mucositis expresses far more receptors for hyaluronic acid than normal tissue. When the composition is applied to the mucositis, because of the lesser viscosity of the first fraction, the composition spreads throughout the mucositis and both fractions bind to the RHAMM and CD44 receptors expressed, taking the COX-2 inhibitor, if used, and penetrate into and throughout the inflamed tissue to ease the patient's pain and aid in the healing of the mucositis. Because the first and second fractions are anti-inflammatory (non-inflammatory) and non-toxic, they do not adversely affect the mucositis treatment with the pharmaceutical composition. The inhibitor in the amount used is non-toxic.

Thus, the selection of the first fraction form of hyaluronic acid is such as not to include fractions which are inflammatory causing. For example, when the first fraction has a molecular weight between about 1200 Daltons and about 5000 Daltons, the first fraction preferably does not include a form of hyaluronic acid having a molecular weight below about 1000 Daltons which has inflammatory activity.

Examples of inflammatory/non-inflammatory forms of hyaluronic acid are discussed in “Size Matters: Molecular Weight Specificity of Hyaluronan Effects in Cell Biology”, International Journal of Cell Biology, Volume 2015, ID 563818, 8 pages, Cyphert et al. Others are known in the art.

See, for example, “A Systematic Study of the Effect of Different Molecular Weights of Hyaluronic Acid on Mesenchymal Stromal Cell-Mediated Immodulation”, which discusses the non-inflammatory effect of Hyaluronic Acid having about 19 disaccharide units which corresponds to a molecular weight of 7.5 kDaltons (7500 Daltons). The authors are Gómez-Aristizábal A, Kim K-P, Viswanathan S (2016), and the article is published in PLoS ONE 11(1): e0147868.

doi:10.1371/journal.pone.0147868.

A formulation which we propose to be suitable for the treatment of mucositis, such as oral mucositis, may comprise:

sterile water, sodium hyaluronate (about 7.5 kiloDaltons) as 0.5 percent of the formulation, sodium hyaluronate (about 650 kiloDaltons) as 2.5 percent of the formulation, diclofenac sodium as 3 percent of the formulation, an effective solubilizing excipient for the diclofenc, and benzyl alcohol excipient.

The following procedure may be used to manufacture a suitable pharmaceutical composition:

3% Diclofenac in 0.5% sodium hyaluronate (molecular weight (i) between about 2000 and about 5000 Daltons or (ii) between about 16000 and about 20000 Daltons or (iii) about 7.5 kDaltons) non-inflammatory first fraction, and 2.5% sodium hyaluronate (molecular weight between about 150000 and about 750000 Daltons) non-inflammatory second fraction, Gel Sterile Water Baxter AW456K 1200 ml-Benzyl Alcohol BDH 23797 15G (14 ml), 3% Diclofenac Sodium, solubilizer for sodium diclofenac, 0.5%, 2.5% Sodium Hyaluronate, HTL Biotech, MW about 679000, 0.5% sodium hyaluronate, HTL Biotech.

Procedure

Set up stirring apparatus using a 2 liter stainless steel beaker,

Add water, solubilizer for diclofenac sodium, and Benzyl Alcohol and stir for 20 minutes to mix,

Add Diclofenac Sodium and stir for 30 minutes to dissolve,

Add both amounts of Hyularonate Sodium slowly and stir initially at a high speed, but avoid splashing,

After addition, stir at a slower speed for 90 minutes, the slower speed reduces the formation of air bubbles,

The result is a clear transparent, viscous gel which is poured into jars and tubes.

Once again, instructions accompany the container and where applicable appropriate devices for providing a premeasured amount of the composition accompany the container.

One form of hyaluronic acid and/or pharmaceutically acceptable salts thereof (for example, sodium salt) and fragments, and sub-units of hyaluronic acid, preferably hyaluronic acid and pharmaceutically acceptable salts thereof, suitable for use with Applicant's invention as the second fraction, is a fraction supplied by HTL Biotech.

The sodium hyaluronate amount is a 2.5% solution with a mean average molecular weight of about 679000 Daltons. The fraction also contains water q.s. which is triple distilled and sterile in accordance with the U.S.P.

The following references teach hyaluronic acid, sources thereof, and processes for the manufacture and recovery thereof which may be suitable so long as they are essentially anti-inflammatory (non-inflammatory):

U.S. Pat. No. 4,141,973 teaches hyaluronic acid fractions (including sodium salts) having:

“(a) an average molecular weight greater than about 750,000, preferably greater than about 1,200,000—that is, a limiting viscosity number greater than about 1400 cm.sup.3/g., and preferably greater than about 2000 cm.sup.3/g.;

(b) a protein content of less than 0.5% by weight;

(c) ultraviolet light absorbance of a 1% solution of sodium hyaluronate of less than 3.0 at 257 nanometers wavelength and less than 2.0 at 280 nanometers wavelength;

(d) a kinematic viscosity of a 1% solution of sodium hyaluronate in physiological buffer greater than about 1000 centistokes, preferably greater than 10,000 centistokes;

(e) a molar optical rotation of a 0.1-0.2% sodium hyaluronate solution in physiological buffer of less than −11.times.10.sup.3 degree-cm.sup.2/mole (of disaccharide) measured at 220 nanometers;

(f) no significant cellular infiltration of the vitreous and anterior chamber, no flare in the aqueous humour, no haze or flare in the vitreous, and no pathological changes to the cornea, lens, iris, retina, and choroid of the owl monkey eye when one milliliter of a 1% solution of sodium hyaluronate dissolved in physiological buffer is implanted in the vitreous replacing approximately one-half the existing liquid vitreous, said HUA being

(g) sterile and pyrogen free and

(h) non-antigenic.”

Canadian Letters Patent 1,205,031 (which refers to U.S. Pat. No. 4,141,973 as prior art) refers to hyaluronic acid fractions having average molecular weights of from 50,000 to 100,000; 250,000 to 350,000; and 500,000 to 730,000 and discusses processes of their manufacture.

Suppliers of the forms of hyaluronic acid are known to the person skilled in the art. The forms of course, as would be known to persons skilled in the art, must be medical grade to be used to treat mucositis. The fractions must also be non-inflammatory (anti-inflammatory). Some suppliers are referred to in U.S. Pat. No. 5,972,906.

As suitable pharmaceutical formulations may be made and used with many variations without departing from the scope of the invention, it is intended that all material contained herein be interpreted as illustrious of the invention and not be interpreted in a limiting sense.

1-50. (canceled)

51. A pharmaceutical composition suitable for use to treat mucositis (including oral mucositis) in an animal (human) suffering from mucositis, said composition comprising an anti-inflammatory (non-inflammatory) first fraction of a form of
exogenous hyaluronic acid selected from hyaluronic acid, a pharmaceutically acceptable salt thereof, a pharmaceutically acceptable ester thereof, a fraction thereof and a subunit thereof, which are anti-inflammatory (non-inflammatory), and
an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons,
the first fraction form of hyaluronic acid haying a lower molecular weight than the second fraction form of hyaluronic acid,
and the first fraction having a lower viscosity than the second fraction.

52. The pharmaceutical composition of claim 51 wherein the molecular weight of the anti-inflammatory (non-inflammatory) form of hyaluronic acid of the first fraction is selected from a form of hyaluronic acid having a molecular weight between about
(1200) 2000 and about 5000 Daltons, a form of hyaluronic acid having a molecular weight about 7.5 kDaltons and a form of hyaluronic acid having a molecular weight between about 16000 and about 20000 Daltons, and combinations of the forms which together are anti-inflammatory (non inflammatory).

53. The pharmaceutical composition of claim 51 further comprising a pharmaceutically tolerable non-toxic amount of COX-1 and or COX-2 inhibitor which is not a form of hyaluronic acid.

54. The pharmaceutical composition of claim 52 further comprising a pharmaceutically tolerable non-toxic amount of COX-1 and/or COX-2 inhibitor which is not a form of hyaluronic acid.

55. The pharmaceutical composition of claim 53 wherein the inhibitor is selected from diclofenac, diclofenac sodium and another pharmaceutically tolerable salt of diclofenac.

56. The pharmaceutical composition of claim 54 wherein the inhibitor is selected from diclofenac, diclofenac sodium, and another pharmaceutically tolerable salt of diclofenac.

57. The pharmaceutical composition of claim 51 wherein the first fraction is between about 0.5 percent and about 3 percent of the composition and the second fraction is between about 2 percent and about 4 percent of the composition.

58. The pharmaceutical composition of claim 52 wherein the first fraction is between about 0.5 percent and about 3 percent of the composition and the second fraction is between about 2 percent and about 4 percent of the composition.

59. The pharmaceutical composition of claim 53 wherein the first fraction is between about 0.5 percent and about 3 percent of the composition and the second fraction is between about 2 percent and about 4 percent of the composition.

60. The pharmaceutical composition of claim 53 wherein the inhibitor is selected from diclofenac and a pharmaceutically non-toxic salt of diclofenac including diclofenac sodium and includes a taste masker.

61. The pharmaceutical composition of claim 54 wherein the inhibitor is selected from diclofenac and a pharmaceutically non-toxic salt of diclofenac including diclofenac sodium and includes a taste masker.

62. The pharmaceutical composition of claim 55 wherein the inhibitor is selected from diclofenac and a pharmaceutically non-toxic salt of diclofenac including diclofenac sodium and includes a taste masker.

63. The pharmaceutical composition of claim 56 wherein the inhibitor is selected from diclofenac and a pharmaceutically non-toxic salt of diclofenac including diclofenac sodium a taste masker.

64. The pharmaceutical composition of claim 59 wherein the inhibitor is selected from diclofenac and a pharmaceutically non-toxic salt of diclofenac including diclofenac sodium and includes a taste masker.

65. The pharmaceutical composition of claim 53 wherein the inhibitor comprises between about 2½ percent and about 4 percent of the pharmaceutical composition.

66. The pharmaceutical composition of claim 54 wherein the inhibitor comprises between about 2½ percent and about 4 percent of the pharmaceutical composition.

67. The pharmaceutical composition of claim 55 wherein the inhibitor comprises between about 2½ percent and about 4 percent of the pharmaceutical composition.

68. The pharmaceutical composition of claim 56 wherein the inhibitor comprises between about 2½ percent and about 4 percent of the pharmaceutical composition.

69. The pharmaceutical composition of claim 59 wherein the inhibitor comprises between about 2½ percent and about 4 percent of the pharmaceutical composition.

70. The pharmaceutical composition of claim 60 wherein the inhibitor comprises between about 2½ percent and about 4 percent of the pharmaceutical composition.

71. The pharmaceutical composition of claim 61 wherein the inhibitor comprises between about 2½ percent and about 4 percent of the pharmaceutical composition.

72. The pharmaceutical composition of claim 62 wherein the inhibitor comprises between about 2½ percent and about 4 percent of the pharmaceutical composition.

73. The pharmaceutical composition of claim 63 wherein the inhibitor comprises between about 2½ percent and about 4 percent of the pharmaceutical composition.

74. The pharmaceutical composition of claim 64 wherein the inhibitor comprises between about 2½ percent and about 4 percent of the pharmaceutical composition.

75. A method of treating mucositis in a human, the method comprising the application of an effective amount of a pharmaceutical composition directly to the site of the mucositis thereby to treat the mucositis, the pharmaceutical composition comprising an anti-inflammatory (non-inflammatory) first fraction of a form of exogenous hyaluronic acid selected from hyaluronic acid, a pharmaceutically acceptable salt thereof, a pharmaceutically acceptable ester thereof, a fraction thereof and a subunit thereof, which are anti-inflammatory (non-inflammatory), and an anti-inflammatory (non-inflammatory) second fraction of exogenous hyaluronic acid selected from hyaluronic acid, a pharmaceutically acceptable salt thereof, a pharmaceutically acceptable ester thereof, a fraction thereof, and a subunit thereof, the form of hyaluronic acid in the second fraction having a molecular weight between about 150000 and about 750000 Daltons, the first fraction form of hyaluronic acid having a lower molecular weight than the second fraction form of hyaluronic acid, and the first fraction having a lower viscosity than the second fraction.

76. The method of treatment of claim 75 wherein the molecular weight of the anti-inflammatory (non-inflammatory) form of hyaluronic acid of the first fraction is selected from a form of hyaluronic acid having a molecular weight between about 2000 and about 5000 Daltons, a form of hyaluronic acid having a molecular weight about 7.5 kDaltons and a form of hyaluronic acid having a molecular weight between about 16000 and about 20000 Daltons, and combinations of the two forms which together are anti-inflammatory (noninflammatory).

77. The method of treatment of claim 75 further comprising a pharmaceutically tolerable non-toxic amount of COX-1 and/or COX-2 inhibitor which is not a form of hyaluronic acid.

78. The method of treatment of claim 76 further comprising a pharmaceutically tolerable non-toxic amount of COX-1 and/or COX-2 inhibitor which is not a form of hyaluronic acid.

79. The method of treatment of claim 77 wherein the inhibitor is selected from diclofenac, diclofenac sodium and another pharmaceutically tolerable salt of diclofenac.

80. The method of treatment of claim 78 wherein the inhibitor is selected from diclofenac, diclofenac sodium, and another pharmaceutically tolerable salt of diclofenac.

81. The method of treatment of claim 75 wherein the first fraction is between about 0.5 percent and about 3 percent of the composition and the second fraction is between about 2 percent and about 4 percent of the composition.

82. The method of treatment of claim 76 wherein the first fraction is between about 0.5 percent and about 3 percent of the composition and the second fraction is between about 2 percent and about 4 percent of the composition.

83. The method of treatment of claim 77 wherein the first fraction is between about 0.5 percent and about 3 percent of the composition and the second fraction is between about 2 percent and about 4 percent of the composition.

84. The method of treatment of claim 77 wherein the inhibitor is selected from diclofenac and a pharmaceutically non-toxic salt of diclofenac including diclofenac sodium.

85. The method of treatment of claim 78 wherein the inhibitor is selected from diclofenac and a pharmaceutically non-toxic salt of diclofenac including diclofenac sodium the method of treatment of claim 79 wherein the inhibitor is selected from diclofenac and a pharmaceutically non-toxic salt of diclofenac including diclofenac sodium.

86. The method of treatment of claim 79 wherein the inhibitor is selected from diclofenac and a pharmaceutically non-toxic salt of diclofenac including diclofenac sodium.

87. The method of treatment of claim 80 wherein the inhibitor is selected from diclofenac and a pharmaceutically non-toxic salt of diclofenac including diclofenac sodium and further includes s taste masker.

88. The method of treatment of claim 83 wherein the inhibitor is selected from diclofenac and a pharmaceutically non-toxic salt of diclofenac including diclofenac sodium and further includes s taste masker.

89. The method of treatment of claim 77 wherein the inhibitor comprises between about 2½ percent and about 4 percent of the pharmaceutical composition.

90. The method of treatment of claim 78 wherein the inhibitor comprises between about 2½ percent and about 4 percent of the pharmaceutical composition.

91. The method of treatment of claim 79 wherein the inhibitor comprises between about 2½ percent and about 4 percent of the pharmaceutical composition.

92. The method of treatment of claim 80 wherein the inhibitor comprises between about 2½ percent and about 4 percent of the pharmaceutical composition.

93. The method of treatment of claim 83 wherein the inhibitor comprises between about 2½ percent and about 4 percent of the pharmaceutical composition.

94. The method of treatment of claim 84 wherein the inhibitor comprises between about 2½ percent and about 4 percent of the pharmaceutical composition.

95. The method of treatment of claim 85 wherein the inhibitor comprises between about 2½ percent and about 4 percent of the pharmaceutical composition.

96. The method of treatment of claim 86 wherein the inhibitor comprises between about 2½ percent and about 4 percent of the pharmaceutical composition.

97. The method of treatment of claim 87 wherein the inhibitor comprises between about 2½ percent and about 4 percent of the pharmaceutical composition.

98. The method of treatment of claim 87 wherein the inhibitor comprises between about 2½ percent and about 4 percent of the pharmaceutical composition.

99. The method of treatment of claim 87 wherein the inhibitor comprises between about 2½ percent and about 4 percent of the pharmaceutical composition.
